(19) United States
(12) Patent Application Publication
(10) Pub. No.: US 2017/0212048 A1
(43) Pub. Date: Jul. 27, 2017

(54) ANALYTE SENSING SYSTEM AND METHOD UTILIZING SEPARATE EQUILIBRIUM AND MEASUREMENT CHAMBERS

(71) Applicants: Govind RAO, Ellicott City, MD (US); Yordan KOSTOV, Columbia, MD (US); Xudong GE, Ellicott City, MD (US); Leah TOLOSA, Columbia, MD (US); Hung LAM, Baltimore, MD (US); Robert HENDERSON, Baltimore, MD (US)

(72) Inventors: Govind RAO, Ellicott City, MD (US); Yordan KOSTOV, Columbia, MD (US); Xudong GE, Ellicott City, MD (US); Leah TOLOSA, Columbia, MD (US); Hung LAM, Baltimore, MD (US); Robert HENDERSON, Baltimore, MD (US)

(21) Appl. No.: 13/823,897

(22) PCT Filed: Sep. 30, 2011
(86) PCT No.: PCT/US11/54434

§ 371 Date: Feb. 16, 2016

Related U.S. Application Data
(60) Provisional application No. 61/388,219, filed on Sep. 30, 2010.

(51) Int. Cl. G01N 21/64; G01N 33/18; G01N 31/22; G01N 21/78

(52) U.S. Cl. CPC G01N 21/643; G01N 21/78; G01N 33/1886; G01N 31/223; G01N 2021/7786

(57) ABSTRACT

An analyte sensing system is provided that utilizes separate and decoupled equilibrium and measurement chambers for improved sensitivity and stability. The system and method are particularly suited for monitoring of CO₂ levels in oceans or other bodies of water.

### GOVERNMENT RIGHTS

This invention was made with government support under Grant No. ONR N00014-10-1-0243 awarded by the Office of Naval Research. The government may have certain rights in this invention.

### BACKGROUND OF THE INVENTION

This application claims priority to U.S. Provisional Application Ser. No. 61/388,219, filed Sep. 30, 2010, whose entire disclosure is incorporated herein by reference.

1. Field of the Invention

The present invention relates to analyte sensing systems and, more particularly, to an analyte sensing system and method with separate equilibrium and measurement chambers for improved sensitivity and stability.

2. Background of the Related Art

Pollution is one of the major problems in the modern industrialized world. In the United States, industry is the greatest source of pollution, accounting for more than half the volume of all water pollution and for the most deadly pollutants. Some 370,000 manufacturing facilities use huge quantities of freshwater to carry away wastes of many kinds. The waste-bearing water is discharged into streams, lakes, or oceans. In its National Water Quality Inventory, the U.S. Environmental Protection Agency concluded that approximately 40% of the nation's surveyed lakes, rivers, and estuaries were too polluted for such basic uses as drinking supply, fishing, and swimming. Pollution not only increases the concentrations of harmful substances, but also changes the pH and the levels of CO₂ and oxygen dissolved in a body of water, thereby disrupting the water's ecological balance, killing off some plant and animal species while encouraging the overgrowth of others.

Another environmental problem is global warming. The majority of scientists studying climate changes believe that global warming is likely caused by increasing amount of greenhouse gases (mostly CO₂) discharged into the environment by human activity. Global warming has numerous deleterious effects, such as rising sea levels, changing the amount and pattern of precipitation, and increasing the intensity of extreme weather events and changing agricultural yields. The rise in CO₂ levels, while contributing to global warming, is also creating ocean acidification at an alarming rate as the oceans collect more CO₂. Warming water and increasingly acidic seas will further change the global ecosystem.

Scientists studying the association between Earth climate and extinctions stated that “the global temperatures predicted for the coming centuries may trigger a new ‘mass extinction event’, where over 50 percent of animal and plant species would be wiped out.” Many of the species especially at risk are Arctic and Antarctic fauna because those species usually rely on cold weather conditions to survive and to reproduce. This will adversely affect the existing local fisheries upon which humans depend. Additionally, it has been shown that climate change due to increases in carbon dioxide concentration is largely irreversible for 1,000 years after emissions have stopped. Concerted action is required to minimize the effect of global warming and most national governments have signed and ratified the Kyoto Protocol aimed at reducing greenhouse gas emissions.

Meanwhile, governments are in the process of implementing requirements for systems to monitor pCO₂ levels and for alleviating the severity of the effects of global warming. Therefore, there is currently an urgent need for small, sensitive, highly stable, low-cost, calibration-free pCO₂ sensing systems for ocean monitoring.

### SUMMARY OF THE INVENTION

An object of the invention is to solve at least the above problems and/or disadvantages and to provide at least the advantages described hereinafter.

Therefore, an object of the present invention is to provide a system and method for sensing analytes.

Another object of the present invention is to provide a system and method for sensing analytes in which the sample containing the analyte being measured does not interfere with the measurement of the analyte.

Another object of the present invention is to provide a system and method for sensing CO₂ in ocean water or in other bodies of water.

To achieve at least the above objects, in whole or in part, there is provided an analyte sensing system, comprising an equilibrium chamber adapted to receive a sensing reagent and a medium containing one or more analytes to be measured, wherein the sensing reagent is adapted to chemically interact and/or physically react with the one or more analytes to be measured, a diffusion membrane in the equilibrium chamber positioned between the medium and the sensing agent, wherein the diffusion membrane is adapted to allow one or more analytes to diffuse from the medium to the sensing reagent, a measurement chamber coupled to the equilibrium chamber for receiving the sensing agent after one or more analytes have diffused from the medium to the sensing reagent, and a detection system for analyzing the sensing reagent in the measurement chamber in order to detect the one or more analytes that diffused into the sensing reagent.

To achieve at least the above objects, in whole or in part, there is also provided an analyte sensing system, comprising an equilibrium chamber having a reagent section for receive a sensing reagent and a sample section for receiving a sample containing one or more analytes to be measured, wherein the sensing reagent is adapted to chemically interact and/or physically react with the one or more analytes to be measured, a diffusion membrane in the equilibrium chamber positioned between the reagent section and the sample section, wherein the diffusion membrane is adapted to allow one or more analytes to diffuse from the sample section to the reagent section, a measurement chamber coupled to the equilibrium chamber for receiving the sensing agent after one or more analytes have diffused from the sample to the sensing reagent, and a detection system for analyzing the sensing reagent in the measurement chamber in order to detect the one or more analytes that diffused into the sensing reagent.

To achieve at least the above objects, in whole or in part, there is also provided a method of measuring at least one analyte, comprising the steps of directing a sample containing the at least one analyte to be measured to an equilibrium chamber containing sensing reagent and a diffusion membrane such that the at least one analyte to be measured diffuses through the diffusion membrane and into the sensing reagent, directing the sensing reagent containing the at least one analyte to be measured to a separate measurement chamber; and analyzing the sensing reagent in the measurement chamber in order to detect the one or more analytes that diffused into the sensing reagent.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIGS. 1 is a schematic cross-sectional side view of a fluorescence-based sensor;

FIG. 2 is a block diagram that illustrates the principles of operation of one preferred embodiment of the analyte sensing system of the present invention;

FIG. 3 is a schematic diagram of an analyte sensing system in accordance with another embodiment of the present invention;

FIGS. 4A is a top plan view of an equilibrium chamber embodiment that can be used in the analyte sensing system of FIG. 3, in accordance with the present invention;

FIG. 4B is a schematic side view of the equilibrium chamber of FIG. 4A looking along the view line A-A of FIG. 4A;

FIG. 4C is a bottom plan view of the equilibrium chamber of FIG. 4A;

FIG. 5 is a perspective view of another equilibrium chamber embodiment that can be used in the analyte sensing system of FIG. 3, in accordance with the present invention;

FIG. 6 is a schematic diagram of a measurement chamber embodiment that can be used in the analyte sensing system of FIG. 3, in accordance with the present invention;

FIG. 7A is a top plane view of another measurement chamber embodiment that can be used in the analyte sensing system of FIG. 3, in accordance with the present invention and

FIG. 7B is a cross-sectional view of the measurement chamber of FIG. 7A taken along the section line B-B.

### DETAILED DESCRIPTION OF PREFERRED EMBODIMENTS

The present invention is particularly suitable for fluorescence-based sensors, such as, for example, fluorescence-based sensors for ocean pCO₂ monitoring. Therefore, the present invention will be primarily described and illustrated in connection with a pCO₂ monitor designed for monitoring of CO₂ levels in oceans or other bodies of water. However, the present invention is also applicable to any reagent-based sensor system for sensing any type of analyte.

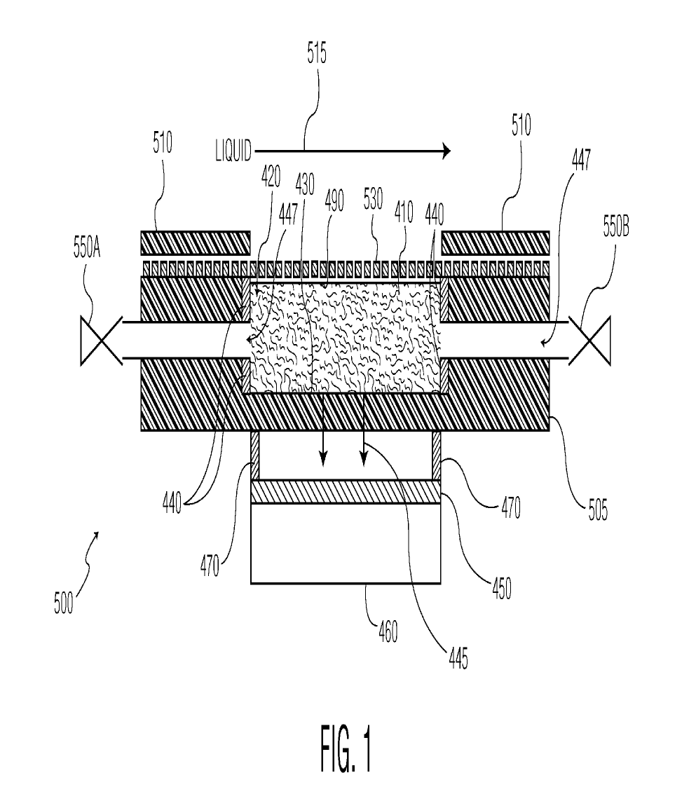

FIG. 1 is a schematic cross-sectional side view of a fluorescence-based sensor 500 with a combined equilibrium and measurement chamber, which is described in co-pending, commonly assigned U.S. patent application Ser. No. 12/692,752, which is incorporated herein by reference in its entirety.

The sensor 500 includes a liquid fluorescence-based sensing reagent 410 that is contained in a recessed cavity 420. The housing 505 of the sensor 500 has a transparent bottom surface 430 that transmits fluorescence light from the fluorescence-based sensing reagent 410, and reflective sides 440 for reflecting unabsorbed excitation light (not shown) back towards the fluorescence-based sensing reagent 410. The reflective sides 440 allow for more uniform excitation of the fluorescence-based sensing reagent 410 by the excitation light. All sides of the recessed cavity 420 are preferably reflective, except for openings 447 that allows new sensing reagent 410 to be pumped into and out of the recessed cavity via valves 550A and 550B.

The resulting fluorescence 445 propagates through the transparent bottom surface 430 to emission filter 450, which is preferably a band-pass filter that passes wavelengths of 550 nm±20 nm. The filtered fluorescence light is detected by detector 460, which is preferably a photodiode. The detector 460 is preferably shielded from outside light by a barrier 470, which is preferably formed from a black material (e.g., black plastic, anodized aluminum, etc.) and attached to the transparent bottom surface 430 and the emission filter 450, suitably with temporary glue or held in place with mechanical means (e.g., clamp, elastic band, etc.).

The recessed cavity 420 is preferably cylindrically-shaped. This geometry maximizes the uniformity of the distribution of excitation light in the fluorescence-based sensing reagent 410.

A lid 510 keeps the diffusion membrane 530 in place. Optical isolation can optionally be provided for the sensing reagent 410, preferably in the form of a thin white filter paper layer 490 under the diffusion membrane 480. The housing 505 in which the mirrored recessed cavity 420 is formed is preferably transparent to the fluorescence wavelengths of the sensing reagent 410. Although the sensor 500 is particularly suited for monitoring of oceans or other large bodies of water, it can also be used for monitoring of O₂, CO₂ or other parameters in a liquid medium of a bioreactor. In addition, although the sensing reagent 410 has been described as a liquid reagent, a gas sensing reagent can also be used.

The housing 505 and lid 510 are preferably made of transparent material, such as poly(methylmethacrylate), PETG, polystyrene, etc. The lid 510 is preferably attached to the diffusion membrane 480 and the housing 505 by any transparent pressure-sensitive adhesive. It can be silicone-based or acrylic-based (e.g., 9770 adhesive from 3M). The reflective sides 440 of the recessed cavity 420 are preferably formed by silvering them using Tolen's reaction (the same process used in making silver mirrors on glass or plastic substrates).

As discussed above, sensor 500 is particularly suited for the measurement of a parameter in a liquid medium 515, such as ocean water, lake water or any other liquid medium and its operation will be described in this context below. However, the sensor 500 may be adapted as any type of fluorescence-based sensing system such as, for example, a O₂ sensing system or a CO₂ sensing system. The light source (not shown), fluorescence-based sensing reagent 410 and emission filter 450 are chosen based on the analyte being measured. The system 500 can also be adapted and used to measure both O₂ and CO₂.

Some preliminary research has been done to identify a fluorescent dye with the best properties for ocean monitoring. Three pH sensitive fluorescent dyes, 8-hydroxypyrene-1,3,6-trisulfonic acid trisodium salt (HPTS, pKa=7.3), 6,8-dihydroxy-1,3-pyrenedisulfonic acid disodium salt (DHDS, pKa=7.3), and 5-(and-6)-carboxynaphthofluorescein mixed isomers (CNF, pKa=7.6) have been compared. The measurements were conducted using a Cary Eclipse (Varian) laboratory spectrometer.

Although all three of the dyes have approximately the same sensitivity to pCO₂ and can achieve a similar precision, DHDS tends to polymerize and precipitate in carbonate buffered saline while CNF has a poor solubility in neutral pH or acidic solutions. Because stability is one of most important considerations for autonomous ocean sensor, HPTS is also preferably used as the fluorescent dye for CO₂ sensing applications.

In operation, a pump (not shown), is programmed to input fresh aliquot of sensor reagent 410 from a reservoir (not shown) into the recessed cavity 420 via valve 550A. A CO₂ diffusing membrane 530 (or a membrane designed to diffuse whatever other analyte is being measured) is stretched over the recessed cavity 420 to permit CO₂ from the ocean or other body of water 515 to diffuse into the recessed cavity 420 and affect a change in the excitation spectrum. A waste chamber collection bottle (not shown) is used downstream to collect spent sensing reagent 410 that is discharged via valve 550B. The two valves 550A and 550B are also used to seal off the recessed cavity 420 during measurements. A bubble trap (not shown) may be employed if necessary. A controller (not shown) may be programmed to control the pump to inject new sensing reagent 410 into the recessed cavity 420 at predetermined time intervals.

During operation of the sensor 500, the recessed cavity 420 is preferably flushed with 200 μL of the sensing reagent 410 twenty minutes before each measurement and the fluorescence intensities are measured as soon as possible to get an initial reading. Preferably, twenty minutes later, after CO₂ diffusion across the membrane 530 and equilibrium is reached, the fluorescence intensities are measured again. From the changes in the fluorescence intensities, the pCO₂ level in the water 515 can be calculated. The actual life of each “batch” of sensing reagent 410 can be measured and replaced once drift exceeds specifications. When a fresh “batch” of sensing reagent 410 is generated after exhaustion of the previous one, very little to no drift at all is expected.

The design of the sensor 500 exhibits some disadvantages due to the equilibrium and measurement chambers being combined. For example, because the sea water from which the analyte (e.g., CO₂) originates is in such close proximity to the excitation light and optical detectors, highly fluorescent molecules in the sea water may be excited by the excitation light that may leak through the diffusion membrane 530. The fluorescence from the molecules in the sea water may introduce noise into the measurement of the fluorescence 445 from the sensing reagent 410. Variations in ocean color due to algae and/or organic matter exacerbates this problem.

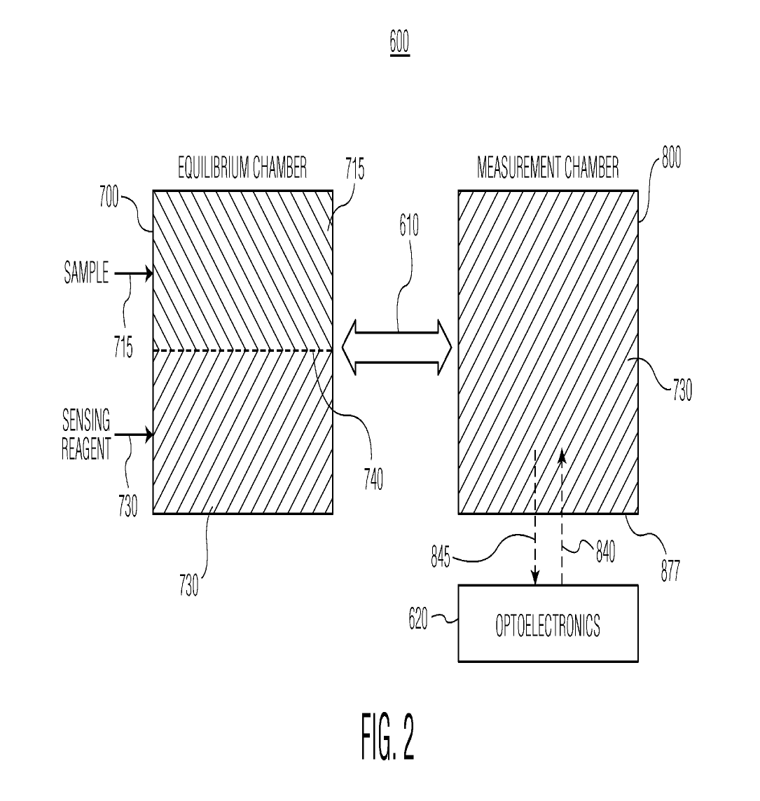

FIG. 2 is a block diagram that illustrates the principles of operation of one preferred embodiment of the present invention. The system 600 includes an equlibrium chamber 700 and a measurement chamber 800 that are coupled via coupling components 610. The equilibrium chamber 700 is adapted to receive and contain a sensing reagent 730 and a sample 715 containing an analyte to be measured. The sample 715 and the sensing reagent are separated by a diffusion membrane 740. The diffusion membrane 740 is chosen based on the analyte to be measured. For example, if the system 600 is designed for the measurement of CO₂ present in a sample 715 (such as a sample of ocean water), then a diffusion membrane 740 is chosen that will allow for the diffusion of CO₂ from the sample 715 into the sensing reagent 730.

The sensing reagent 730 can be a liquid reagent or a gas reagent. If the system 600 is designed to be used with a liquid sensing reagent 730, then the coupling components 610 can include any combination of hydraulic components such as, for example, valves, conduits, pumps, intake ports, exit ports, etc. If the system 600 is designed to be used with a gas sensing reagent 730, then the coupling components 610 can include any combination of pneumatic components such as, for example, valves, conduits, pumps, intake ports, exhaust ports, etc.

The sensing reagent 730 is preferably an optical chemical sensing reagent that exhibits at least one optical property (e.g., light emission intensity, light absorption, etc.) that is dependent on the amount of the analyte that is being sensed and/or monitored. The system 600 also preferably includes optoelectronics 620 for exciting the sensing reagent with excitation light 840 and for receiving emission light 845 from the sensing reagent 730. To this end, at least one surface 847 of the measurement chamber 800 is transparent to the optical excitation light 840 and the emission light 845, and the optoelectronics are positioned to direct the optical excitation light 840 through the transparent surface 847 and to receive emission light 845 that propagates through the transparent surface 847.

In operation, a sample 715 (e.g., a sample of ocean water) which contains the analyte being measured is directed to the equilibrium chamber 700. The analyte to be measured diffuses across the diffusion membrane 740 into the sensing reagent 730. After equilibrium is established, the sensing reagent 730 is then transferred from the equilibrium chamber 700 to the measurement chamber 800 using coupling components 610 in order to measure the analyte that diffused into the sensing reagent 730 using optoelectronics 620.

Once the sensing reagent 730 is transferred to the measurement chamber 800, fluorescence measurements can commence using optoelectronics 620. Because the equilibrium chamber 700 and the measurement chamber 800 are decoupled, auto-fluorescence of the sample 715 will not factor into the measurement because the sample 715 is not present in the measurement chamber 800.

The sample 715 that contains the analyte being measured can be a liquid sample, such as a sample of ocean water or lake water, or a gas sample. Similarly, the sensing reagent 730 can be either a liquid reagent or a gas reagent that can be transferred to the measurement chamber using coupling components 610.

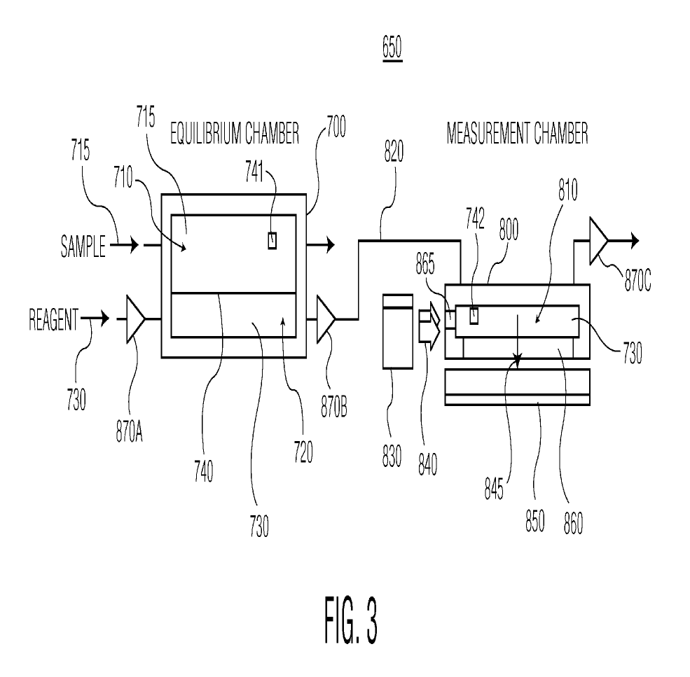

FIG. 3 is a schematic diagram of an analyte sensing system 650, in accordance with another embodiment of the present invention. The system 650 includes an equilibrium chamber 700 and a separate measurement chamber 800. The equilibrium chamber 700 includes a sample section 710 for receiving a sample 715 containing an analyte to be measured, and a reagent section 720 for containing a sensing reagent 730 such as, for example, a fluorescent-based sensing reagent. A diffusion membrane 740 separates the sample section 710 from the reagent section 720. The diffusion membrane 740 is chosen based on the analyte to be measured. For example, if the system 600 is designed for the measurement of CO₂ present in a sample 715 (such as a sample of ocean water), then a diffusion membrane 740 is chosen that will allow for the diffusion of CO₂ into the reagent section 720.

The measurement chamber 800 includes a reagent cavity 810 for receiving the sensing reagent 730 from the equilibrium chamber 700 via conduit 820. In a preferred embodiment, the sensing reagent 730 is a fluorescent-based sensing reagent and the system 600 also includes an optical excitation source 830 for generating excitation light 840 that is used to optically excite the sensing reagent 730. Examples of optical excitation sources include, but are not limited to, light emitting diodes (LEDs) and laser diodes. The measurement chamber 800 and the equilibrium chamber 700 may optionally include respective heaters 741 and 742 that can be independently controlled for independent control of temperature in each chamber.

The system 650 also preferably includes an optical detector 850, preferably a photodetector, for the detecting the light emission 845 (e.g., fluorescence) from the sensing reagent 730. At least one of the walls that define the reagent cavity 810 has a transparent portion 860 that is transparent to the light emitted by the sensing reagent 730 in response to the excitation light 840. Similarly, at least second transparent portion 865 is formed in one of the walls that define the reagent cavity 810 that is transparent to the optical excitation light 840 from the optical excitation source 830.

The system 650 includes valves 870A, 870B and 870C for controlling the flow of sensing reagent 730 into the equilibrium chamber 700, from the equilibrium chamber 700 to the measurement chamber 800, and out of the measurement chamber 800 once the measurement has been completed. In operation, valve 870A is opened and sensing reagent 730 is pumped into the reagent section 720. Once the sensing reagent 730 is pumped into the reagent section 720, valve 870A is closed to seal off the reagent chamber 720.

The sample 715 containing the analyte to be measured is directed to the sample section 710. Although the sample section 710 is shown as an enclosed section in FIG. 3, the sample section could be open to the environment so that the equilibrium chamber can be placed in a medium (e.g., a body of water or gas environment) containing the analyte to be measured (e.g., CO₂), such that the medium comes into contact with the diffusion membrane 740. Generally, any design known in the art can be used for the equilibrium chamber 700 as long as a sample 715 containing the analyte to be measured comes into contact with the diffusion membrane 740 that separates the sample section 710 from the reagent section 720. The reagent section 740 must be physically isolated from the sample 715 containing the analyte to be measured such that diffusion of the analyte into the sensing reagent 730 only occurs through the diffusion membrane 740.

The analyte to be measured diffuses across the diffusion membrane 740 into the reagent 730. After equilibrium is established, the sensing reagent 730 is then pumped from the equilibrium chamber 700 to the measurement chamber 800 via conduit 820 for by opening valve 870B. The conduit 820 can be any means know in the art for transferring a liquid or gas such as, for example, plastic tubing (e.g., Tygon® tubing).

Once the sensing reagent 730 is pumped into the reagent cavity 810 of the measurement chamber 800, fluorescence measurements can commence using the excitation source 830 and optical detector 850. Because the equilibrium chamber 700 and the measurement chamber 800 are decoupled, auto-fluorescence of the sample 715 will not factor into the measurement because the sample is not present in the measurement chamber 800. The sample section 710 and reagent section 720 of the equilibrium chamber 700 can each be of any shape, as long as they are separated by a diffusion membrane 740.

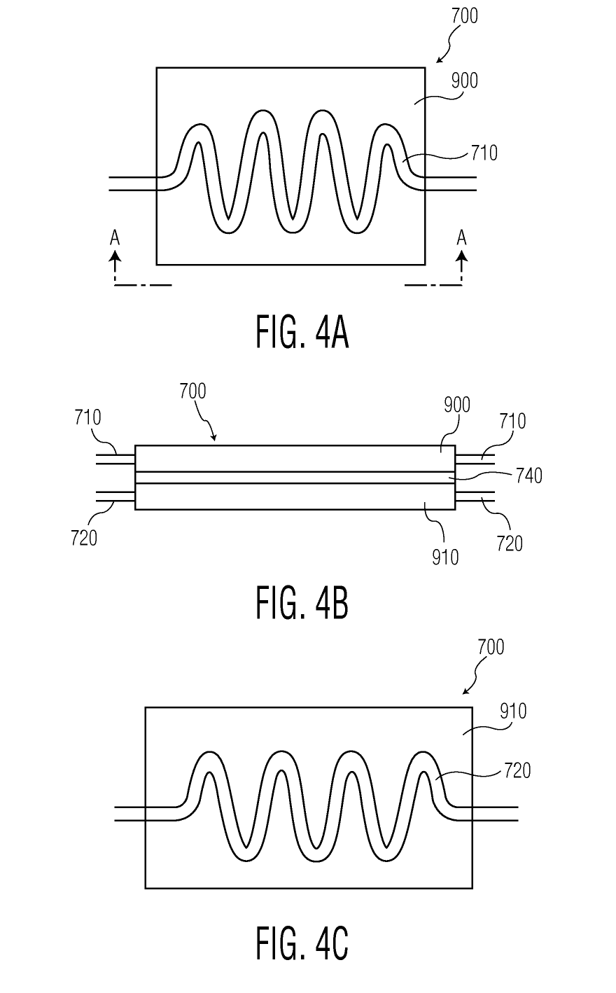

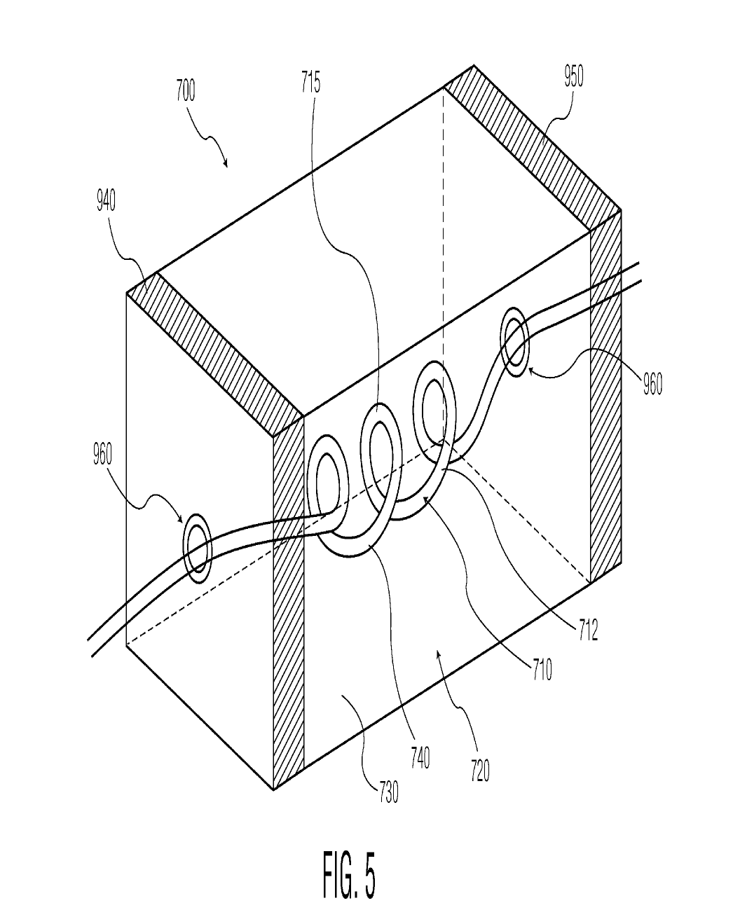

For example, FIGS. 4A-4C show an equilibrium chamber 700 that includes serpentine-shaped sample and reagent sections 710 and 720, respectively. The equilibrium chamber of FIGS. 4A-4C include a sample section 710 that is a serpentine-shaped channel formed on a first substrate 900. Similarly, the reagent section 720 a channel formed on a second substrate 910 with the same serpentine shape as the sample section 710. The diffusion membrane is sandwiched between the first and second substrates 900 and 910, such that the serpentine-shaped channels that form the sample and reagent sections 710 and 720 substantially overlap each other. The substrates 900 and 910 are attached to each other, with the diffusion membrane 740 sandwiched between them, with any adhesive known in the art.

In the equilibrium chamber embodiment of FIGS. 4A-4C, the sensing reagent 730 flows through the serpentine-shaped reagent section 720 and the sample 715 flows through the serpentine-shaped sample section 710. Diffusion of the analyte to be measured takes place from the sample section 710 to the counterpart, similarly shaped reagent section 720 via the diffusion membrane 740.

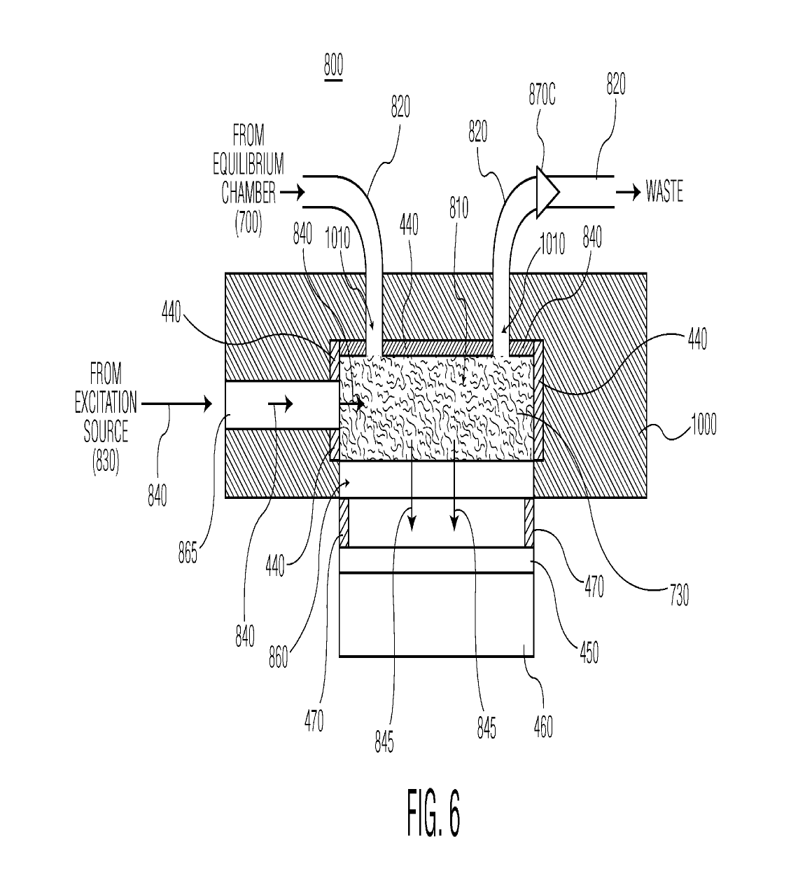

FIG. 5 shows an equilibrium chamber 700 in accordance with another embodiment of the present invention. The equilibrium chamber 700 includes a reagent section 720, and a sample section 710 that comprises tubing 712 that is passed through the reagent section 720. The tubing 712 that makes up the sample section 710 can be any type of tubing known in the art that is permeable to the analyte being measured so that it can function as the diffusion membrane 740 between the sample 715 in the sample section 710 and the sensing reagent 730 in the reagent section 720.

The tubing 712 is preferably flexible so that a larger amount of tubing can be placed inside the reagent section 720. The more tubing 712 is present in the reagent section 720, the more analyte diffusion can take place between the tubing 712 and the reagent 730 due to the larger tubing surface area in contact with the reagent 730. End portions 940 and 950 of the equilibrium chamber 700 contain openings 960 for the tubing 712 to enter and exit the equilibrium chamber 700. Although the equilibrium chamber 700 is shown as rectangularly-shaped, it can be any other shape, such as cylindrically-shaped. Further, the equilibrium chamber 700 can suitably be a flow cell that has been modified to pass the tubing 712 through the reagent section 720.

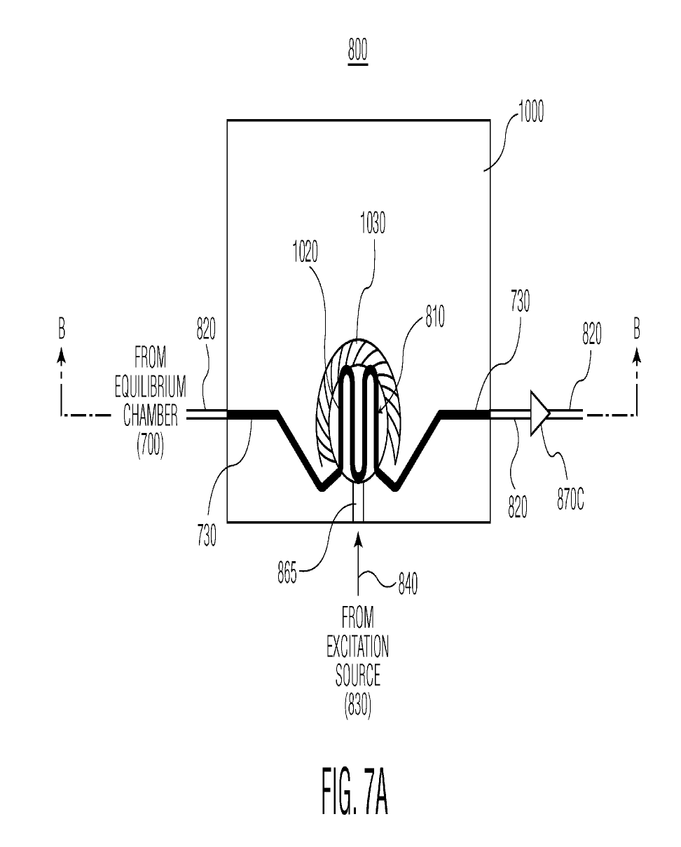

FIG. 6 is a schematic diagram of a measurement chamber 800 that can be used in the analyte sensing system 650 of FIG. 3, in accordance with one embodiment of the present invention. The measurement chamber 800 includes a reagent cavity 810 for holding the sensing reagent 730 received from the equilibrium chamber 700 via conduit 820. The housing 1000 of the measurement chamber 800 has a transparent bottom portion 860 that transmits fluorescence light 845 from the sensing reagent 730, and reflective sides 440 for reflecting unabsorbed excitation light 840 back towards the sensing reagent 730. The reflective sides 440 allow for more uniform excitation of the sensing reagent 730 by the excitation light 840. All sides of the reagent cavity 810 are preferably reflective, except for openings 1010 that allows new sensing reagent 730 to be pumped into and out of the reagent cavity 810, transparent portion 860, and transparent portion 865 that allows excitation light 840 into the reagent cavity 810.

The resulting fluorescence light 845 propagates through the transparent portion 860 to emission filter 450, which is preferably a band-pass filter that passes wavelengths corresponding to the fluorescence light 845. The filtered fluorescence light is detected by detector 460, which is preferably a photodiode. The detector 460 is preferably shielded from outside light by a barrier 470, which is preferably formed from a black material (e.g., black plastic, anodized aluminum, etc.) and attached to the transparent bottom surface 860 and the emission filter 450, suitably with temporary glue or held in place with mechanical means (e.g., clamp, elastic band, etc.).

The reflective sides 440 of the reagent cavity 810 are preferably formed by silvering them using Tolen's reaction (the same process used in making silver mirrors on glass or plastic substrates). The reagent cavity 810 is preferably cylindrically-shaped. This geometry increasing the uniformity of the distribution of excitation light 840 in the sensing reagent 730. However, any other shape can be used for the reagent cavity while still falling with the scope of the present invention.

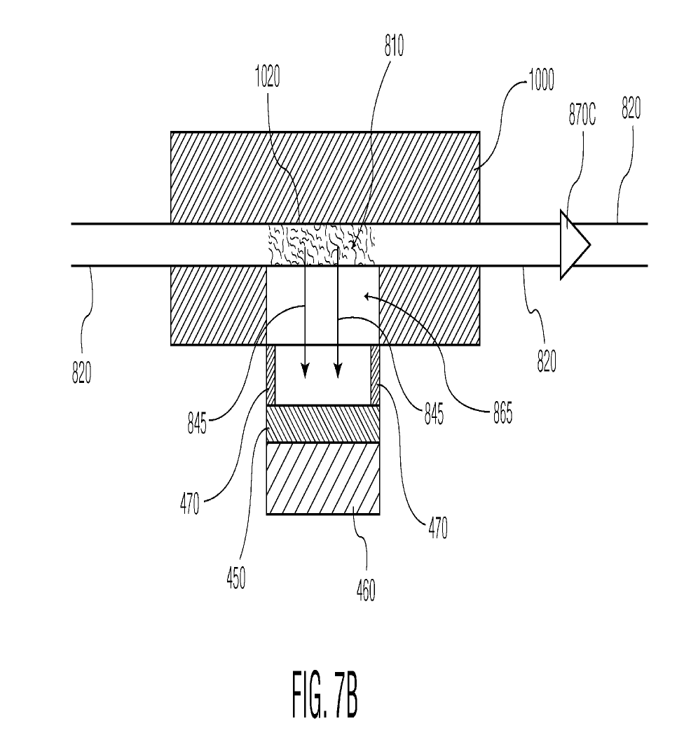

For example, FIGS. 7A and 7B are schematic top and cross-sectional side views, respectively, of another preferred embodiment for a measurement chamber 800 that can be used in the analyte sensing system 650 of FIG. 3. In the embodiment of FIG. 7, the reagent cavity 810 is implemented with a serpentine-shaped channel 1020 formed in the housing 1000 of the measurement chamber 800.

As with the embodiment of FIG. 6, the housing 1000 contains a transparent portion 865 for transmitting excitation light 840 from the optical excitation source (not shown in FIG. 7). The channels 1020 are transparent to the wavelength of the optical excitation light 840 and also transparent to the wavelength of the fluorescence emission light 845 emitted by the sensing reagent 730.

As with the embodiment of FIG. 6, sending reagent is pumped into and out of the reagent cavity 810 formed by the channels 1020 via conduits 820. An integrating mirror 1030 is formed in the housing and substantially surround the reagent cavity 810 for increasing the uniformity of the distribution of excitation light 840 in the sensing reagent 730.

The fluorescence emission light 845 is preferably detected in the same manner as that shown in the FIG. 6 embodiment. That is, the fluorescence emission light 845 from the reagent cavity 810 (serpentine channels 1020) propagates through the transparent portion 860 to emission filter 450, which is preferably a band-pass filter that passes wavelengths corresponding to the fluorescence light 845. The filtered fluorescence light is detected by detector 460, which is preferably a photodiode. The detector 460 is preferably shielded from outside light by a barrier 470, which is preferably formed from a black material (e.g., black plastic, anodized aluminum, etc.) and attached to the transparent bottom surface 860 and the emission filter 450, suitably with temporary glue or held in place with mechanical means (e.g., clamp, elastic band, etc.).

The foregoing embodiments and advantages are merely exemplary, and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. Various changes may be made without departing from the spirit and scope of the invention, as defined in the following claims. For example, although the present analyte sensor system has been predominantly described in connection with a CO₂ sensing system, it can be applied to any fluorescence based sensing system. Further, although the present analyte sensor system has been described as being particularly suited for monitoring large bodies of water, such as oceans and lakes, they can be used to monitor analytes in any type of liquid or gas media, such as liquid or gas media inside a bioreactor.

What is claimed is:

1. An analyte sensing system, comprising:
an equilibrium chamber adapted to receive a sensing reagent and a medium containing one or more analytes to be measured, wherein the sensing reagent is adapted to chemically interact and/or physically react with the one or more analytes to be measured;
a diffusion membrane in the equilibrium chamber positioned between the medium and the sensing agent, wherein the diffusion membrane is adapted to allow one or more analytes to diffuse from the medium to the sensing reagent;
a measurement chamber coupled to the equilibrium chamber for receiving the sensing agent after one or more analytes have diffused from the medium to the sensing reagent; and
a detection system for analyzing the sensing reagent in the measurement chamber in order to detect the one or more analytes that diffused into the sensing reagent.

2. The system of claim 1, wherein the sensing agent comprises a fluorescence-based sensing reagent.

3. The system of claim 1, wherein the medium containing one or more analytes to be measured comprises ocean water and the one or more analytes comprise CO₂.

4. The system of claim 2, wherein the detection system comprises:
at least one optical excitation source for optically exciting the fluorescence-based sensing reagent in the measurement chamber; and
at least one optical detector for detecting fluorescence emission light from the fluorescence-based sensing reagent in the measurement chamber.

5. The system of claim 1, wherein the sensing reagent comprises a liquid.

6. The system of claim 5, wherein the equilibrium chamber and the measurement chamber are coupled with hydraulic coupling components.

7. The system of claim 6, wherein the hydraulic coupling components comprise a first conduit for hydraulically connecting the equilibrium chamber to the measurement chamber, wherein the first conduit comprises at least one valve for controlling the flow of the sensing reagent between the equilibrium chamber and the measurement chamber.

8. The system of claim 7, further comprising:
a second conduit for selectively introducing the sensing reagent into the equilibrium chamber, wherein the second conduit comprises at least one valve for controlling the flow of the sensing reagent into the equilibrium chamber; and
a third conduit for selectively removing the sensing reagent from the measurement chamber, wherein the third conduit comprises at least one valve for controlling the flow of the sensing reagent out of the measurement chamber.

9. An analyte sensing system, comprising:
an equilibrium chamber having a reagent section for receive a sensing reagent and a sample section for receiving a sample containing one or more analytes to be measured, wherein the sensing reagent is adapted to chemically interact and/or physically react with the one or more analytes to be measured;
a diffusion membrane in the equilibrium chamber positioned between the reagent section and the sample section, wherein the diffusion membrane is adapted to allow one or more analytes to diffuse from the sample section to the reagent section;
a measurement chamber coupled to the equilibrium chamber for receiving the sensing agent after one or more analytes have diffused from the sample to the sensing reagent; and
a detection system for analyzing the sensing reagent in the measurement chamber in order to detect the one or more analytes that diffused into the sensing reagent.

10. The system of claim 9, wherein the sensing agent comprises a fluorescence-based sensing reagent.

11. The system of claim 9, wherein the sample containing one or more analytes to be measured comprises ocean water and the one or more analytes comprise CO₂.

12. The system of claim 10, wherein the detection system comprises:
at least one optical excitation source for optically exciting the fluorescence-based sensing reagent in the measurement chamber; and
at least one optical detector for detecting fluorescence emission light from the fluorescence-based sensing reagent in the measurement chamber.

13. The system of claim 9, wherein the sensing reagent comprises a liquid.

14. The system of claim 13, wherein the equilibrium chamber and the measurement chamber are coupled with hydraulic coupling components.

15. The system of claim 14, wherein the hydraulic coupling components comprise a first conduit for hydraulically connecting the equilibrium chamber to the measurement chamber, wherein the first conduit comprises at least one valve for controlling the flow of the sensing reagent between the equilibrium chamber and the measurement chamber.

16. The system of claim 15, further comprising:
a second conduit for selectively introducing the sensing reagent into the equilibrium chamber, wherein the second conduit comprises at least one valve for controlling the flow of the sensing reagent into the equilibrium chamber; and
a third conduit for selectively removing the sensing reagent from the measurement chamber, wherein the third conduit comprises at least one valve for controlling the flow of the sensing reagent out of the measurement chamber.

17. The system of claim 9, wherein the equilibrium chamber comprises:
a first substrate;
a second substrate; and
a diffusion membrane positioned between the first and second substrates.

18. The system of claim 17, wherein the sample section comprises a serpentine-shaped channel formed on the first substrate.

19. The system of claim 18, wherein reagent section comprises a serpentine-shaped channel formed on the second substrate, wherein the first and second serpentine-shaped channels substantially overlap each other.

20. The system of claim 9, wherein the equilibrium chamber comprises a flow cell.

21. The system of claim 20, wherein the sample section comprises tubing that within the flow cell and the diffusion membrane comprises the tubing material.

22. The system of claim 9, wherein the measurement chamber comprises a reagent cavity for receiving reagent from the equilibrium chamber.

23. The system of claim 22, wherein the reagent cavity is serpentine-shaped.

24. A method of measuring at least one analyte, comprising the steps of:
directing a sample containing the at least one analyte to be measured to an equilibrium chamber containing sensing reagent and a diffusion membrane such that the at least one analyte to be measured diffuses through the diffusion membrane and into the sensing reagent;
directing the sensing reagent containing the at least one analyte to be measured to a separate measurement chamber; and
analyzing the sensing reagent in the measurement chamber in order to detect the one or more analytes that diffused into the sensing reagent.

25. The method of claim 24, wherein the sensing reagent comprises a liquid.

26. The method of claim 24, wherein the sensing reagent comprises a fluorescence-based sensing reagent.

27. The method of claim 26, wherein the sensing reagent in the measurement chamber is analyzed by:
exciting the sensing reagent with excitation light; and
measuring fluorescence emission light emitted from the sensing reagent in response to the excitation light.
